(19) United States
(12) Patent Application Publication
(10) Pub. No.: US 2026/0346454 A1
(43) Pub. Date: Nov. 5, 2026

(54) Cobotic System for Intraoperative Data Collection and Predictive Analytics

(71) Applicant: Peritas AI Inc., Fort Lauderdale, FL (US)

(72) Inventor: Martin Roche, Fort Lauderdale, FL (US)

(73) Assignee: Peritas AI Inc., Fort Lauderdale, FL (US)

(21) Appl. No.: 19/426,242

(22) Filed: Dec. 19, 2025

Related U.S. Application Data
(63) Continuation-in-part of application No. 19/195,731, filed on Apr. 30, 2025.

(51) Int. Cl. B25J 9/16; A61B 34/32; A61B 34/35; A61B 90/00; B25J 11/00; B25J 13/00; B25J 18/02; G06T 11/00; G06V 10/75; G06V 10/764; G06V 20/40; G06V 20/58; G06F 3/16; G06V 10/147; G10L 15/22; G10L 21/0208

(52) U.S. Cl. CPC B25J 9/1689; A61B 34/32; A61B 34/35; A61B 90/03; B25J 9/1676; B25J 9/1697; B25J 11/0005; B25J 13/003; B25J 18/025; G06T 11/00; G06V 10/75; G06V 10/764; G06V 20/40; G06V 20/58; A61B 2090/365; G06F 3/167; G06V 10/147; G10L 15/22; G10L 2015/223; G10L 21/0208

(57) ABSTRACT

A perioperative humanoid cobot system includes a robotic platform configured to autonomously support surgical teams through integrated vision, communication, and documentation capabilities. The system comprises a humanoid robotic body with articulated arms and a head-mounted stereoscopic vision system capable of optical, thermal, and depth imaging. A control system coordinates autonomous navigation, personnel and object recognition, sterile field compliance, and intraoperative data acquisition. A multimodal communication interface supports real-time telepresence, enabling remote participants to interact with surgical staff through audio, video, and augmented or virtual reality interfaces. The system further includes a data recording module configured to capture and log timestamped surgical activity, instrument usage, and personnel movements to cloud-based storage for postoperative analysis. Modular components, including swappable sensor payloads and configurable display systems, allow the cobot to adapt to varied surgical workflows while maintaining safe and sterile operation.

### CROSS-REFERENCE TO RELATED APPLICATIONS

This patent application is a continuation-in-part of US Patent Application Ser. No. 19/195,731 filed on Apr. 30, 2025, which claims the benefit of priority to U.S. Provisional Patent Application No. 63/640,524, filed on Apr. 30, 2024, the entirety of which is incorporated herein by refer

### TECHNICAL FIELD

The present invention relates generally to robotic systems in surgical environments, and more specifically to a humanoid perioperative cobot equipped with multiple sensors.

### BACKGROUND

Modern operating rooms are increasingly incorporating telepresence and digital communication tools to enhance surgical collaboration, education, and documentation. Surgeons and medical staff sometimes rely on remote presence robots or video conferencing systems to consult with off-site experts and to observe procedures in real time. Likewise, surgical teams use various imaging devices and recording systems to document operations for training or quality control purposes. However, existing surgical telepresence and communication solutions remain limited and fragmented, failing to fully address the unique challenges of the perioperative environment.

One limitation in current technology is the lack of fully autonomous humanoid robots specifically designed for surgical environments. Present remote presence robots are typically wheeled video-conferencing units or simple mobile carts that require direct human control. These devices generally lack anthropomorphic design and do not possess the autonomy or adaptability needed to navigate a busy operating room independently. They cannot, for example, dynamically reposition themselves with human-like agility or respond on their own to changes in the surgical workflow. As a result, conventional telepresence robots offer only basic remote viewing and voice communication, without the ability to actively participate in or adjust to the intraoperative context.

Moreover, situational awareness and sterile-field compliance are major shortcomings of current telepresence systems. Existing mobile robots have limited sensor capabilities and typically cannot discern critical spatial boundaries such as the sterile field in an operating room. They often depend on remote operators to guide them and lack the real-time environmental understanding to avoid obstacles, maintain proper distances from the surgical site, or respect no-go zones. This limited spatial interaction means that a telepresence device might inadvertently come too close to sterile instruments or personnel, creating contamination risks, or it may be kept at a safe perimeter that reduces its usefulness. In essence, current systems do not intelligently perceive or adapt to the complex, crowded, and sensitive physical environment of surgery.

In addition, the functions of visual data acquisition, instrument tracking, and communication are handled by disparate systems in today’s operating rooms. For instance, high-resolution surgical video may be captured by overhead cameras or endoscopic equipment; instrument location and usage are monitored (if at all) by separate tracking software or manual counting; and communication with remote consultants or team members occurs via standalone audio-video links or basic telepresence units. These fragmented solutions are not integrated with one another, forcing surgical staff to juggle multiple devices and interfaces. Important contextual information can be lost when systems do not share data. For example, a remote observer may see the surgical field through a camera but lack any real-time overlay identifying instruments or personnel, and they cannot easily obtain a comprehensive view of the room’s status or equipment layout. This fragmentation leads to inefficiencies and blind spots in both remote collaboration and intraoperative documentation.

Because of these limitations, current telepresence and recording setups can also pose workflow inefficiencies and potential safety concerns. Setting up separate cameras or communication tools during a procedure may interrupt the surgical flow or require extra personnel. The remote expert’s ability to assist is often constrained by fixed camera angles and the inability to interact directly with the scene, such as pointing out an anatomical landmark or highlighting a particular instrument in view. Similarly, capturing a complete intraoperative record for training or compliance is challenging when using multiple uncoordinated devices, and critical events might be missed or not synchronized. In some cases, valuable data from the operation, whether visual, auditory, or instrument-related, goes uncollected or uncontextualized, reducing the utility of postoperative analysis. All of these issues underscore that existing systems for remote collaboration or intraoperative recording are suboptimal for the demanding setting of surgery.

Accordingly, there is a recognized need for a more capable perioperative robotic platform that overcomes the above shortcomings. In particular, a humanoid cobot designed for the surgical environment is needed, one that can autonomously navigate and position itself without encroaching on sterile zones, while providing rich situational awareness through advanced sensors. Such a system should seamlessly integrate high-resolution visual monitoring, instrument recognition, personnel identification, and communication functions into a single platform. By combining real-time video and audio communication with augmented or virtual reality overlays and on-board intelligence, the cobot could allow remote participants to view the surgery with immersive clarity and interact with the scene (for example, via virtual pointers or annotations) without disrupting the ongoing procedure. Furthermore, an integrated approach to data acquisition is beneficial and the cobot should be able to capture and synthesize multiple data streams in real time – from wide-angle room scans and equipment checks to focused views of surgical maneuvers – and present this information in a coherent way to both local and remote users.

Therefore, there exists a need for an improved perioperative cobotic system that provides fully autonomous, intelligent telepresence and communication capabilities tailored to surgical workflows. Such a humanoid cobot would fill the gap in current technology by offering a unified solution for real-time remote interaction, comprehensive intraoperative documentation, digital device outputs (e.g. surgical robots), and responsive environmental scanning in the operating room. By addressing the limitations of autonomy, situational awareness, system integration, and data responsiveness, this invention lays a foundation for safer, more efficient, and more informative collaboration during surgical procedures.

### SUMMARY

The present invention relates to a perioperative humanoid cobot system configured to provide real-time telepresence, augmented reality interaction, and autonomous data acquisition within surgical environments. The system includes a humanoid robotic platform with articulated arms, a high-resolution stereoscopic vision system, and a multimodal communication interface enabling both local and remote interaction with surgical staff and intra-operative software systems. One or more cameras are mounted on the head or torso of the robot and are configured to capture high-definition visual data of the operating room and surgical field, with support for optical, thermal, or depth imaging.

The system further includes a wireless communication module configured to support real-time transmission of video, audio, and control signals to and from a remote operator interface. An audio communication system with voice recognition and speech synthesis enables two-way verbal interaction between surgical staff and a remote expert. The system includes modules for instrument recognition, personnel identification, and room layout mapping, allowing it to annotate or overlay real-time data for remote display or augmented reality interfaces.

A control system manages autonomous or semi-autonomous navigation of the robot within the operating room, coordinating obstacle avoidance, sterile field compliance, and camera positioning. The robot may operate in a fully autonomous documentation mode, a collaborative semi-autonomous mode, or a remote teleoperation mode. A data recording module stores timestamped visual and environmental data to a cloud-based platform, enabling surgical activity documentation, workflow analysis, and remote access.

The system may further include a user interface for local control and override, a fail-safe collision prevention mechanism, and integration with hospital information systems for surgical schedule alignment, data tagging, and procedure-specific configuration. In one embodiment, a remote operator can view stereoscopic visual feeds through a head-mounted display or monitor, provide verbal instruction, and control camera position or visual overlays for enhanced collaboration.

### BRIEF DESCRIPTION OF DRAWINGS

The foregoing and other objects, features, and advantages of the invention will become more apparent from the following detailed description, taken in conjunction with the accompanying drawings, in which:

FIG. 1 is a perspective view of a humanoid perioperative cobotic system.

FIG. 2 is a schematic block diagram of the system architecture of the cobotic system.

FIG. 3 illustrates a use case scenario of the cobotic system in the operating room.

FIG. 4 is a flow chart of the control system's logic for navigating the cobotic system.

FIG. 5 is a flow diagram of a remote telepresence interaction utilizing the cobotic system.

FIG. 6 is a flow chart of a data acquisition and recording process used by the cobotic system.

### DETAILED DESCRIPTION

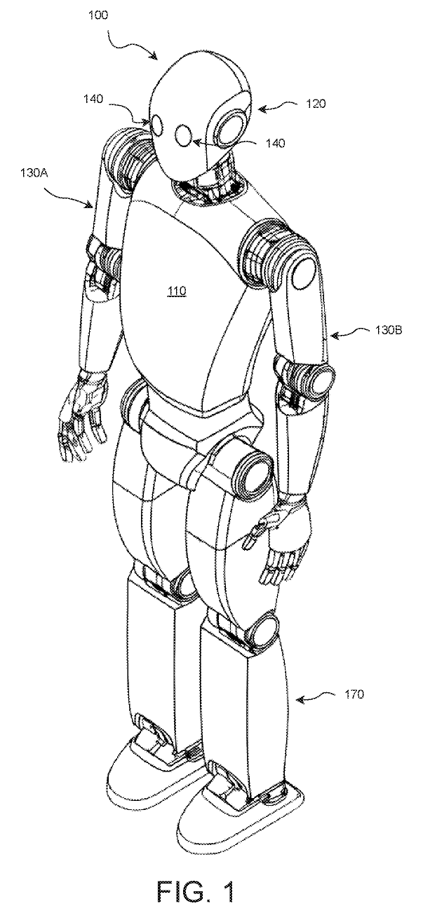

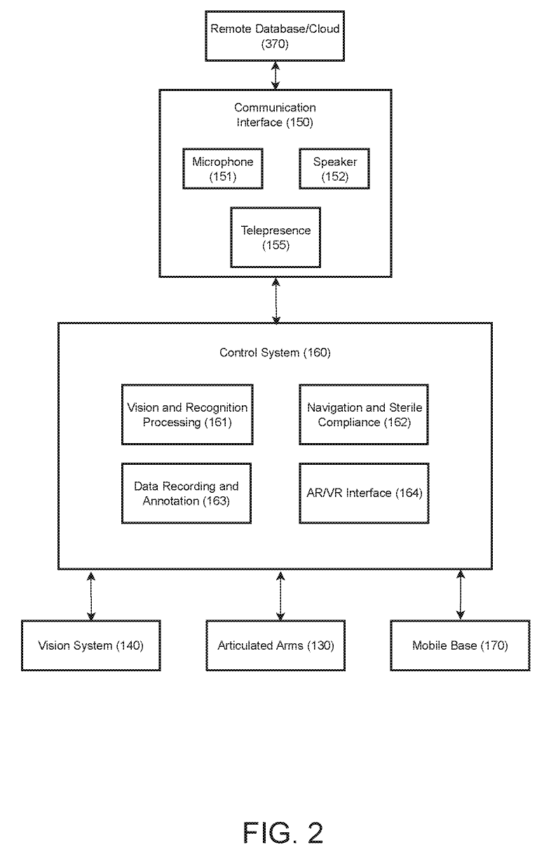

Referring now to the drawings, FIG. 1 illustrates a perspective view of an exemplary perioperative humanoid cobotic system 100 configured to support surgical teams, and FIG. 2 is a block diagram depicting the system’s architecture. The cobotic system 100 has a humanoid form factor including a torso 110 supporting a head 120 and a pair of articulating arms 130. In the embodiment shown, the torso 110 is mounted on a mobile base 170 that provides stability and mobility within an operating room. The mobile base 170 may utilize wheels, tracks, or legs (in various embodiments) driven by integrated actuators to move the cobot 100 around the perioperative environment. The control system 160 (housed within torso 110) regulates the base’s locomotion, enabling the cobot 100 to navigate smoothly and stop or re-position as needed. The humanoid configuration, having a torso 110, head 120, and arm 130 structure, allows the cobot 100 to operate in a manner and scale similar to a human team member, which facilitates intuitive interaction, proper vantage points for observation, and seamless integration into the surgical workflow. It should be noted that while a humanoid form is described as the primary embodiment, the core components of system 100 (vision, communication, control, etc.) can be implemented in other form factors (for example, a cart-mounted unit or overhead gantry) without departing from the scope of the disclosure. However, the humanoid form provides particular advantages in terms of mobility, field of view, and ergonomics when collaborating in a surgical environment.

As shown in FIG. 1, the head 120 of the cobot 100 houses a high-resolution vision system including one or more cameras 140. In one embodiment, the head 120 contains a stereoscopic camera pair 140 oriented forward to capture detailed three-dimensional images of the surgical field, providing depth perception similar to human eyesight. The cameras 140 may be sensitive to visible light and can also incorporate other imaging modalities, such as infrared sensors for low-light or thermal imaging and depth sensors (for example, LiDAR or structured-light cameras), to enhance situational awareness. The vision system is modular: camera units 140 or entire sensor modules can be swapped or upgraded depending on surgical needs (for instance, a module with a higher zoom optical camera or a fluorescent imaging sensor can be attached for specialized procedures). The head 120 can pan, tilt, or otherwise articulate (via internal actuators controlled by control system 160) to adjust the field of view, ensuring that critical surgical activities remain within view even as the team moves. This articulation may mimic human neck movement, allowing the cobot 100 to “look around” the operating room. The arms 130 are likewise articulated and may each have multiple joints (e.g., shoulder, elbow, wrist joints) providing several degrees of freedom. In the embodiment of FIG. 1, two robotic arms 130A, 130B are mounted to the torso 110. These arms 130 can be positioned to avoid interfering with personnel, or utilized to perform auxiliary tasks such as holding additional sensors or tools, adjusting equipment (outside the sterile field), or gesturing to facilitate communication. The arms 130 are designed with safety in mind: they incorporate force/torque sensors and compliant control so that they can operate in close proximity to humans without causing injury, immediately pausing or yielding if even a slight unexpected resistance or contact is detected. The mechanical design of the cobot 100 allows for modularity in hardware: for example, the distal end of an arm 130 may accept interchangeable end-effectors (camera mounts, pointers, or other instruments) to accommodate different support tasks, and the head 120 or sensor suite can be detached and replaced as a unit to introduce new vision capabilities. Key system components, such as the processing hardware of control system 160 or communication interfaces 150, are also modular or upgradeable. For instance, the control system 160 can include swappable computing modules or expansion slots for additional processors, enabling scaling of computational power (e.g., adding a specialized AI co-processor for advanced image analysis or additional memory for data logging) as the complexity of tasks grows. This modularity ensures that the cobot system 100 is adaptable and configurable for a range of perioperative applications and can evolve with technological advances or specific hospital requirements.

With reference to FIG. 2, the internal architecture of the perioperative cobot system 100 will now be described. The control system 160 comprises one or more processors, memory devices, and associated circuitry configured to govern the cobot’s operations. In one embodiment, control system 160 includes a multi-core computing unit dedicated to real-time image processing, robotic control, and data management tasks. The control system 160 is operatively connected to all major subsystems of the cobot 100, including the vision system (cameras 140 and any other sensors), the locomotion drive of mobile base 170, the actuators of arms 130, and the communication interface 150. As shown in FIG. 2, a vision processing module (which may be part of control system 160 or a dedicated sub-module thereof) receives image data from the cameras 140. This vision module leverages advanced computer vision algorithms, which can include machine-learning-based models, to analyze the video and image streams in real time. By processing these data, the system 100 is capable of object recognition and personnel recognition within the operating room. For example, the control system 160 can identify surgical instruments on a tray or in use (distinguishing a scalpel from a hemostat, for instance) and recognize personnel such as the surgeon, anesthesiologist, or nurses, possibly by their faces, attire, or known positional cues. The system 100 can also track the location and movement of these instruments and people over time, creating a dynamic situational model of the OR. This capability not only enables context-aware support (e.g., the cobot 100 can detect when a particular instrument 330 is picked up or a specific staff member 320 enters the room) but also feeds into documentation and safety functions (such as instrument count verification or ensuring non-authorized individuals do not approach the sterile field). The control system 160 further integrates a spatial mapping and localization function: using data from the cameras 140 (including depth data if available) and optionally additional range sensors on the base 170 (like LiDAR or ultrasonic proximity sensors), it continuously generates a three-dimensional map of the operating room environment. This map allows the cobot 100 to know its own position and orientation in the room (localization) and to identify static and moving obstacles, walls, furniture, as well as designated zones such as the sterile field around the patient.

Also depicted in FIG. 2 is the communication interface 150, which encompasses the system’s capabilities for multimodal communication and telepresence. The communication interface 150 includes hardware and software for wireless networking, such as transceivers for Wi-Fi and/or 5G connectivity to ensure robust, low-latency data links. It also includes audio input and output devices (for instance, a microphone 151 and one or more speakers 152 built into the cobot 100). These allow the cobot to capture operating room sounds and speech and to vocalize or play audio in the OR, facilitating two-way voice communication. In some embodiments, the communication interface 150 further includes a video display or projector mounted on the cobot (for example, a screen on the head 120 or torso 110) to visually represent remote participants or to show information to nearby personnel. The system supports voice communication in multiple ways: the surgical team can verbally address the cobot 100 (for instance, giving it instructions or asking it to focus on a particular area) and the cobot’s microphone 151 will pick up these commands for processing by the control system 160 (which may employ speech recognition algorithms). Conversely, the cobot 100 can emit synthesized speech or relay a remote participant’s voice through speaker 152, enabling the remote participant to speak to the local team. The communication interface 150 is operatively coupled to a telepresence module 155 within control system 160 that manages remote connectivity. This module handles the streaming of audio/video data to and from remote devices and ensures secure, encrypted communication to protect patient privacy and comply with medical data security standards.

In the block diagram of FIG. 2, an augmented/virtual reality (AR/VR) interface 164 is also shown as part of the system architecture. The AR/VR interface 164 represents the components (which may be software routines in control system 160, dedicated hardware, or a combination) that generate and manage augmented reality overlays and virtual reality environments for the system 100. Through this interface, the cobot 100 can integrate with AR/VR devices used by local or remote participants. For example, AR/VR interface 164 can format the cobot’s sensor data into a VR environment that a remote user can immerse themselves in (using a VR headset), giving the remote user a virtual presence in the operating room. Likewise, the interface 164 can send processed visual data and annotations to an AR display device used by a surgeon or nurse, such as AR glasses 360 worn by the surgeon or a tablet display, overlaying helpful information onto the surgeon’s view of the real world. The AR overlays might include, for instance, highlighting a particular surgical instrument or anatomical location, showing labels or pointers as guidance from a remote expert, or indicating the boundaries of the sterile field or no-go zones for equipment. The AR/VR interface 164 works in tandem with the vision processing and recognition systems: it can automatically generate visual indicators in AR based on what the cobot 100 observes (for example, flashing a warning overlay if an instrument is detected too close to the edge of the sterile field, or outlining a missing instrument that hasn’t been returned to the tray). Additionally, the interface 180 can incorporate external data sources, for example, pulling in the patient’s vital signs or imaging (like MRI/CT scans) and presenting these as AR visuals at relevant positions to assist the surgical team. The data recording and logging module (not separately numbered in FIG. 2, but part of control system 160) is responsible for storing the data collected by the cobot 100. This includes high-definition video streams from cameras 140, audio recordings, and the metadata gleaned from the object/personnel recognition and system events. It can store data locally (on internal memory or drives in torso 110) and/or transmit data to a secure cloud or hospital server in real-time for archival and further analysis. The entire system architecture is designed with modularity and scalability in mind: new sensors can be added to the vision module, additional communication protocols or interfaces (such as new AR devices or hospital database integrations) can be supported by updating the communication interface 150, and the control system’s software can be updated to incorporate improved algorithms (for example, more advanced machine learning models for recognition or prediction of surgical workflow events). The control system 160 is configured to coordinate all these subsystems seamlessly, enabling the cobot 100 to function as an autonomous assistant that perceives the surgical environment, communicates with human collaborators both local and remote, and maintains a record of the intraoperative data.

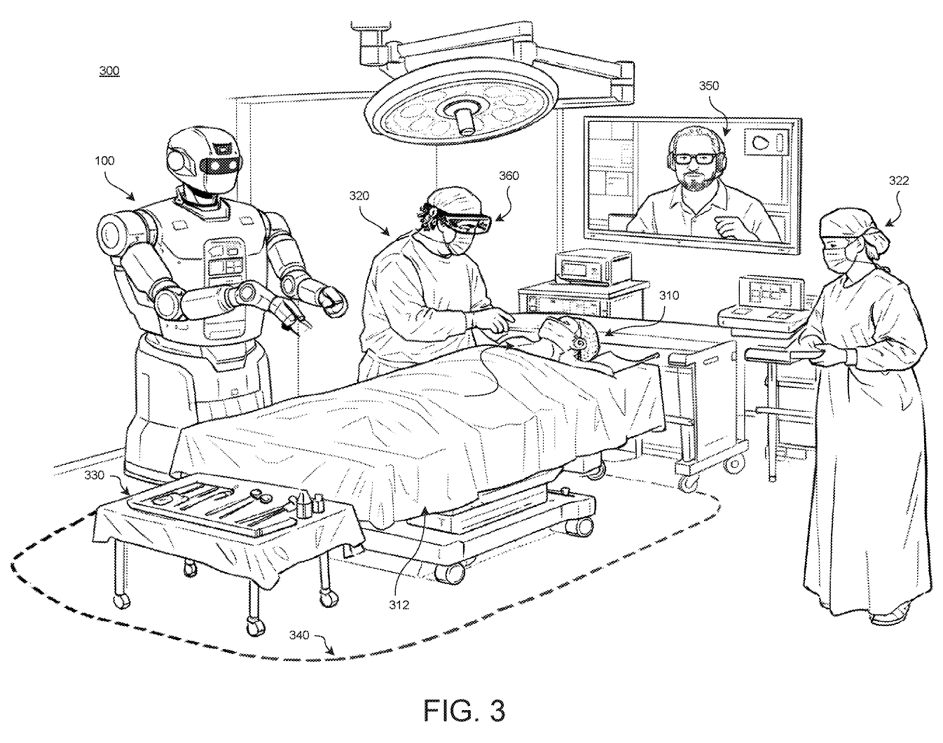

FIG. 3 illustrates an example use-case scenario of the perioperative cobot system 100 in an operating room environment 300, demonstrating collaboration with both local surgical staff and a remote participant. In the diagram, the cobot 100 is positioned at an optimal location in the operating room 300 to observe the surgical field without interfering with the surgical team. For instance, the cobot 100 may be placed just outside the sterile field boundary 340, or inside if sterilely gowned (the designated sterile area immediately surrounding the patient 310 and instrument table) so that it does not risk contaminating sterile surfaces. The torso 110 and articulating head 120 of the cobot are elevated to approximately human head height, allowing the cameras 140 to have an unobstructed line of sight into the operative site on the patient 310 lying on the operating table 312. During the procedure, the cobot’s vision system continuously captures the scene. In this figure, a surgeon 320 and a nurse 322 are working at the operating table, and the cobot 100 is autonomously tracking their movements and actions. The object recognition capabilities enable the system 100 to identify a surgical instrument 330 being used by the surgeon 320, for example, recognizing that the surgeon 320 has picked up a scalpel (instrument 330) from the instrument tray 332. Upon detecting this, the cobot’s control system 160 can automatically log a timestamped event (e.g., “scalpel in use at 10:32:15”) in the surgical record it is compiling. Similarly, the cobot 100 recognizes the presence and roles of personnel: it can distinguish the lead surgeon 320 from the assisting nurse 322 by their physical appearance or attire (optionally cross-referencing with pre-loaded profiles or RFID tags on their ID badges). This information is used both for documentation (noting which personnel were present and active at various times) and for intelligent behavior, for example the cobot 100 can focus its camera on the surgeon’s hands when it identifies that the surgeon 320 is performing a critical task, or it can address individuals specifically if it has a reason to communicate (such as saying “Dr. Smith, a remote consultant would like to speak” directed toward the surgeon).

In the scenario of FIG. 3, the system’s telepresence and AR features are engaged to bring in a remote medical expert 350 to assist or observe the procedure. The remote participant 350 connects to the cobot 100 through a secure network (via the communication interface 150). The remote participant 350 may be using a computing device or telepresence console, which could include a virtual reality headset or other immersive display that presents a live panoramic view of the operating room as captured by the cobot’s cameras 140. Through this interface, the remote expert 350 essentially “sees” from the perspective of the cobot 100 in real time, including the surgeon’s movements and the patient’s status. The audio feed from the operating room (picked up by the cobot’s microphone 151) lets the remote participant hear conversations, patient monitor alarms, and other ambient sounds, providing important context. Conversely, the remote expert 350 can speak, and their voice is transmitted to the cobot 100 and played over the speaker 152 so that the surgical team hears them as if they were in the room. This two-way audio-video link enables true telepresence, the remote expert can offer advice, ask questions, or guide the team just as if they were present in the OR.

The augmented reality aspect of the system is also illustrated: the surgeon 320 in FIG. 3 is shown wearing AR glasses 360 (or alternately viewing a nearby AR-enabled display). These AR glasses 360 are receiving real-time overlay content from the cobot’s AR interface 180. For example, suppose the remote expert 350 wants to highlight an anatomical structure or indicate a direction for an incision. Using their interface, the remote expert can draw a virtual marker or cursor in their VR view. The AR/VR interface 180 in the cobot system will interpret this input and project a corresponding AR overlay into the surgeon’s glasses 360, such as a glowing line superimposed on the patient’s body indicating where the incision should be extended, or a circle around an anatomical feature to be careful of. In another example, the cobot’s own object recognition might detect that a surgical instrument has been left on a surface and not returned to the tray, and automatically generate an AR highlight around that instrument in the surgeon’s view to draw attention to it. The communication interface 150 ensures that these AR annotations and telepresence interactions occur with minimal latency, so that remote guidance feels immediate and synchronized with the live procedure. Throughout this scenario, the cobot 100 remains an autonomous yet cooperative presence: it repositions itself if needed (for instance, quietly rolling a few inches to get a better camera angle when the surgical team changes positions, all the while obeying the constraint of not entering the sterile field 340). It may also use its arms 130 to slightly adjust its built-in camera angle or to gesture, for example, if the remote expert wants to point at something in the physical world, the cobot 100 could extend an arm 130 to gesture toward the indicated object or area (acting as the “hands” of the remote person). Importantly, the cobot’s control system 160 monitors all these actions to ensure safety: it will not move into an area occupied by a person, it keeps a safe distance from the operating table 312, and its arms 130 will not swing in a way that could contact a person or sterile equipment. The result, as depicted in FIG. 3, is a collaborative environment where the humanoid cobot 100 serves as an intelligent assistant and communication conduit, documenting the surgery, providing an extra set of high-tech “eyes” on the field, and extending the surgical team’s capabilities by connecting them with remote expertise and data in real time.

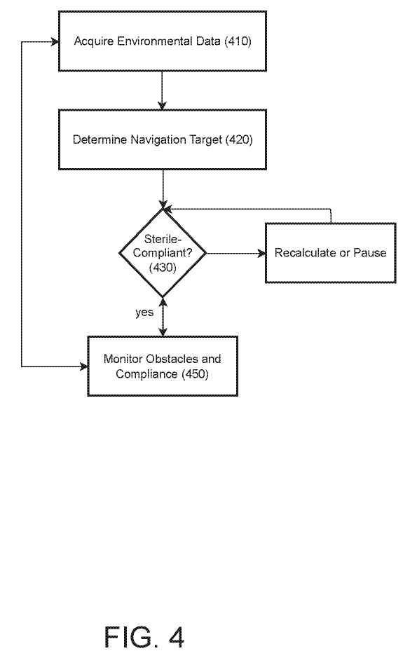

Turning now to FIG. 4, this figure illustrates a flow chart of the control logic that the cobot’s control system 160 employs for autonomous navigation and sterile-field compliance within the operating room. The process depicted in FIG. 4 ensures that the cobot 100 can move about the OR as needed to perform its duties while rigorously avoiding any action that would breach sterile protocol or impede the surgical workflow. The autonomous navigation process begins at step 410, where the cobot 100 (via its cameras 140 and other sensors) acquires data about its surroundings. In this initial step, the control system 160 activates vision and recognition processing 161 and navigation modules 162 to capture a current snapshot of the environment: identifying the locations of nearby objects, the positions of surgical staff, and crucially the boundaries of the sterile field 340. The sterile field may be defined, for example, by the draped area around the patient and instrument table, which the system 100 can recognize either by prior calibration (knowing the coordinates of the table 312 and draped area) or dynamically through image analysis (e.g., detecting the sterile drape color or markers that indicate sterile zones). Additionally, step 410 involves sensing any moving obstacles or changes in the environment since the last update, such as a person walking by or a piece of equipment being moved, using image data and possibly other proximity sensors on the base 170.

At step 420, the control system 160 determines a navigation goal or target position for the cobot 100. This target might be set automatically based on the system’s tasks (for instance, to maintain an optimal view, the cobot may need to move a little to the left if a surgeon is blocking its camera, or reposition to follow a specific staff member or surgical site), or it could be commanded by a user (such as a surgeon saying “come closer” or a remote participant selecting a viewpoint). Once the target location or orientation is identified, the control system 160 proceeds to path planning. Still in step 420, the control system computes a path from the cobot’s current position to the desired target position. This computation uses the environmental map: it plots a trajectory that avoids fixed obstacles (walls, furniture) and gives wide berth to any detected people (to avoid startling or colliding with them). A key aspect at this stage is checking for sterile field intrusion: the planned path is analyzed to see if it would cross into or through the sterile field 340. If the initially calculated path would violate sterile boundaries, the control system 160 adjusts the target or path accordingly, this could mean taking a longer route around the room, or if necessary, not moving at that moment (for example, waiting until a path is clear or the sterile field is reconfigured). The cobot 100 is essentially constrained by virtual no-go zones that correspond to sterile areas; step 430 in the flow chart indicates the decision point where the system determines whether a safe path exists. If no safe path is immediately available (e.g., the only direct route is through the sterile field), the system can either pause and periodically re-check (looping back to step 410 to update sensor data) or request assistance (perhaps alerting the staff that it cannot reach a location without someone adjusting the sterile setup or giving it clearance).

Assuming a valid, sterile-compliant path is found, the process moves to step 440, where the control system 160 issues commands to the cobot’s locomotion actuators to execute the movement. The mobile base 170 then drives the cobot 100 along the planned route. During this movement, the cobot continuously performs obstacle avoidance in real time. As indicated in step 450, the control system monitors the sensor feeds for any new obstacles or changes – for instance, if a nurse 322 unexpectedly steps into the cobot’s path or a piece of equipment is suddenly pushed into the way. If such a dynamic obstacle is detected, the system 160 will immediately adjust: it can slow or stop the cobot 100, recalculate a new path around the obstacle, or if necessary, abort the movement to maintain safety. Importantly, the control logic gives highest priority to avoiding interference with humans and sterile fields; even a minor potential encroachment will cause the cobot to halt or reroute. This responsiveness is possible because the cobot 100 fuses its vision data with rapid control loops governing the motors, allowing it to react within fractions of a second if something or someone comes too close. As part of step 450, the system also enforces speed and distance limits when near the sterile field 340 or personnel, for example, it may automatically reduce speed when within a certain radius of the operating table or a person’s location to ensure gentle, controlled movement. In an embodiment the cobot system 100 may be sterilely gowned to function in the sterile field.

At step 460, having navigated successfully, the cobot 100 arrives at the target position and carries out the intended action (if any) associated with that move. For instance, the target might have been chosen to get a clearer camera angle, so upon reaching it, the cobot 100 might refocus its camera 140 on the surgical site and resume intensive data capture. Another example: the cobot could move to a position to better transmit the surgeon’s view to the remote participant 350, or to position its arm 130 to pick up audio more clearly from a certain person speaking – whatever the goal, step 460 is the fulfillment of that objective. Once in position, the cobot 100 continues to monitor its stability and compliance and it ensures that even when stationary, it does not inadvertently lean or extend into the sterile area, and that its presence is not obstructing OR staff routes. After completing the action at step 460, the process may return to step 410 as an ongoing loop, because the surgical environment is dynamic. The cobot 100 will continuously reassess whether it should remain where it is or navigate to a new position as the procedure progresses. In summary, the flow of FIG. 4 encapsulates an autonomous navigation algorithm that intelligently balances the need for the cobot 100 to reposition and follow the action with the absolute requirement of maintaining sterile integrity and safety. Thanks to this logic, the cobot can operate with minimal supervision, deftly moving around the room to stay out of the way of personnel, never crossing forbidden boundaries, and always positioning itself advantageously to support the surgical team’s needs.

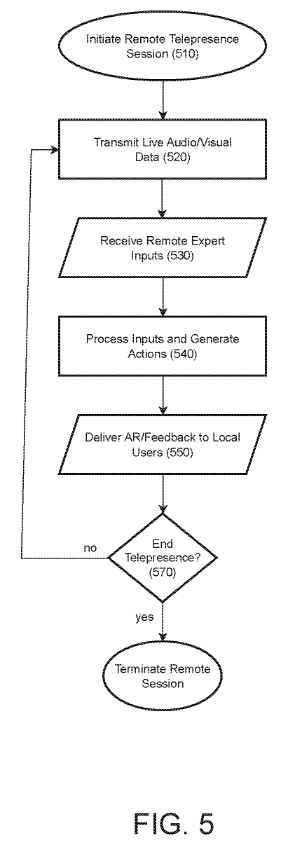

FIG. 5 illustrates a flow chart of the remote telepresence interaction and AR/VR integration process facilitated by the cobot system 100. This process details how a remote participant 350 connects and collaborates via the cobot 100 and how augmented reality features are employed to enhance communication. The process begins at step 510, where a remote session is initiated. In step 510, a remote user (such as a consulting surgeon at a different location) establishes a secure communication link with the perioperative cobot 100 through a telepresence platform. This could be triggered by the remote user logging in and requesting to join the procedure, or by the local team sending an invitation. The communication interface 150 authenticates the remote connection and negotiates streaming parameters to ensure high-quality, low-latency transmission of data. Once the connection is established, the process moves to step 520, in which the cobot 100 transmits live data streams to the remote participant 350. Specifically, the high-resolution video feed from the cobot’s cameras 140 is encoded and streamed, giving the remote participant a real-time view of the operating room (often from the vantage point of the cobot’s head 120). Along with video, audio from the OR microphone 151 is streamed so that the remote user can hear the ongoing conversation among the surgical staff and the ambient sounds of the procedure. Step 520 may also involve sending additional sensory data if available – for example, if the cobot has environmental sensors or if it’s pulling vital sign data from the patient monitor, those data can be forwarded to the remote user’s interface for a comprehensive situational picture.

At step 530, the interaction becomes bi-directional: the remote participant 350 can now actively engage. The remote user’s interface can be, for example a VR headset, a computer with a display, or other interface that captures the remote user’s inputs such as the user’s voice, gestures, or direct manipulations of a virtual environment. For instance, the remote expert might speak a guidance message or ask a question; the audio from the remote side is sent over the network to the cobot 100. Alternatively or additionally, the remote user might use a pointer or controller in their VR space to indicate something on the video feed. These inputs are received by the cobot’s telepresence module (within communication interface 150) and processed at step 530. If the input is audio (the remote person speaking), the cobot 100 will output it through speaker 152 for the surgical team to hear. If the input is an annotation or control command (for example, the remote user might click on the live video to place a marker, or press a button to request the cobot to zoom in or move its camera), the control system 160 interprets these commands accordingly.

Moving to step 540, the cobot 100 acts on the remote participant’s inputs and integrates them into the local environment via augmented reality or direct control. For voice inputs, this simply means continuing the conversation (no AR aspect, just ensuring clarity of audio in the OR). For visual annotations or guidance from the remote user, step 540 involves generating the appropriate AR overlay or indication for the surgical team. For example, if the remote user drew a circle around a tumor margin in their VR view, the AR/VR interface 180 processes that gesture and maps it to the real-world coordinates of the surgical field. The result is that the surgeon’s AR glasses 360 (or other display) now show a highlighted circle or other graphical indicator at the corresponding location on the patient or the display of an endoscopic image, etc. Similarly, if the remote user requests a different camera angle, the control system 160 can pan or tilt the cobot’s head 120, or possibly instruct an arm 130 to reposition an attached camera, thereby complying with the request. At step 550, the augmented reality feedback is delivered to the local users. This means the surgeon 320 and possibly other staff see the annotations or guidance from the remote expert overlaid on their view of the patient or instruments (through AR glasses, a heads-up display, or even on a screen in the OR). If the remote participant 350 is sharing any reference images or data (for instance, pointing to a preoperative scan or showing a comparative diagram), those too can be displayed in the AR device or on a screen such that the surgical team can easily view them without breaking scrub or looking away from the operative field. Step 550 thus represents the synchronized AR collaboration: everyone both locally and remotely has a shared visual context, with the remote participant’s inputs seamlessly integrated into what the local team is seeing.

Throughout steps 530–550, the communication remains interactive and continuous. At step 560, the process emphasizes maintaining the real-time feedback loop. The remote participant 350 receives confirmation or visual feedback that their guidance has been implemented (for instance, they might see on their own display that the surgeon has followed the indicated line or that the cobot’s camera has zoomed as requested). Likewise, the local team can respond – the surgeon might speak back to acknowledge or ask a question, which gets transmitted to the remote user, or the surgeon could use a hand gesture or laser pointer in the OR which the cobot’s cameras pick up so the remote user sees it. The system ensures that these interactions happen with minimal delay, and it manages bandwidth to prioritize the most critical data (for example, maintaining smooth video and audio). If network conditions degrade, the control system 160 may temporarily reduce the video quality or frame rate rather than losing the connection entirely, to ensure continuous communication. Redundant communication channels can be employed (such as a secondary cellular connection) as a fail-safe so that the telepresence link remains active even in the face of connectivity issues.

Finally, at step 570, the telepresence session can be closed or transitioned as needed. This step might involve the remote expert 350 ending their consult and the communication interface 150 securely terminating the data streams. Before ending, the system may prompt to save relevant data from the session, for instance any AR annotations or guidance given could be logged (time-stamped and associated with the video) for post-operative review. Additionally, step 570 can include resetting the AR overlays (clearing any markers from the surgeons’ view) and ensuring the cobot 100 returns to its default autonomous mode after any remote control input (for example, if the remote user had control over the camera, that control is now handed back to the cobot’s autonomous system). The outcome of the process in FIG. 5 is a successful remote interaction: the cobot 100 has functioned as the eyes, ears, and even a virtual pointer for the remote participant, while the AR system bridged the gap between remote guidance and local action, thereby significantly enhancing the collaborative capabilities of the surgical team.

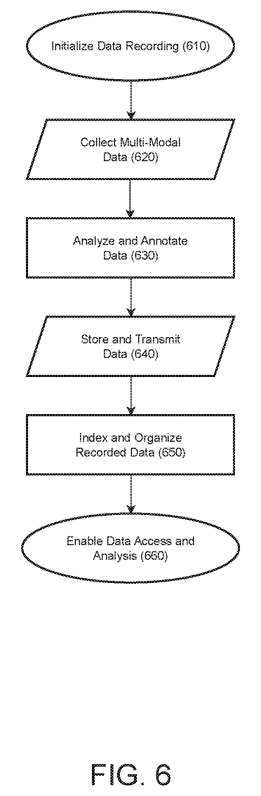

Turning to FIG. 6, a flow chart is shown for the data acquisition and cloud-based recording process executed by the cobot system 100 during perioperative activities. This process details how the system 100 captures, records, and disseminates surgical data in a secure and organized manner for documentation and later use. The process begins at step 610, typically at the start of a surgical procedure (or any perioperative event to be documented). In step 610, the control system 160 initializes the data recording sequence in a data recording and annotation module 163. This can be triggered automatically (for example, by integration with the operating room scheduling system or by detecting that a patient 310 has entered the OR and the surgery is about to begin) or manually by a user command (such as a voice instruction from the surgeon or a prompt on a user interface to “start recording”). Upon initiation, the system timestamps the start of the record and may create a new entry or file in its storage corresponding to this specific procedure or session.

Once recording is initiated, the process proceeds to step 620, where the cobot 100 collects multi-modal data from the operating environment. The primary data streams include the continuous video feed from the high-definition cameras 140, providing a visual record of the surgery from the cobot’s perspective. Alongside video, audio data is captured via the microphone 151, recording spoken communication among the surgical staff, any alarms or sounds from medical devices, and ambient room noise. In some embodiments, the system 100 can also ingest data from other sources at this stage: for example, it might receive a feed of the patient’s vital signs (heart rate, blood pressure, etc.) from the anesthesia monitoring equipment via a wired or wireless interface, or receive outputs from a surgical robot indicative of parameters relevant to a given procedure. It could also gather data from specialized sensors if present (such as environmental sensors measuring room temperature, or additional cameras covering different angles). All these data are synchronized against a common timeline to ensure that events across modalities can be correlated (video and audio are time-aligned, and if an EKG data stream is included, it’s timestamped to match the video frames, etc.).

At step 630, the system 100 performs in-process analysis and annotation of the collected data to enrich the documentation. The control system 160 utilizes its artificial analysis based recognition algorithms (as described earlier with respect to FIG. 2) to interpret what is happening in the video and audio streams. For instance, as the video is being recorded, the system can detect notable events such as “first incision made”, “instrument X picked up or put down”, “surgeon changes position”, or “patient moved”, etc. When such events are detected, the system 100 generates metadata or flags that get associated with the video timeline. For example, if the cobot recognizes that a particular instrument 330 (say a sponge or a needle) is removed and not returned, it can mark that video frame with an annotation indicating a potential surgical count discrepancy. Or if it recognizes a person 320 entering or exiting the room, it can log that with a timestamp (“Circulating nurse exited at 11:07”). Speech recognition may also be applied to the audio stream; important verbal remarks (like the surgeon calling out a sponge count or noting a critical patient status change) can be transcribed or flagged in the record. These annotations serve multiple purposes: they ensure no detail is lost in the voluminous raw data, they make it easier post-operatively to search and review specific moments, and they support real-time decision-making (for example, the system might gently alert the team if an instrument that was taken has not been seen returned after an unusually long interval, as a preventive measure against forgotten sponges, etc.). Step 630 thus reflects the system’s ability to not just passively record, but intelligently document the surgery by combining sensor data with AI-driven insights.

In step 640, the cobot’s data management module proceeds to store and transmit the collected data. The rich set of data (video, audio, annotations, etc.) can be stored locally on the cobot’s on-board storage in real time. Simultaneously, or at intervals, the system 100 uploads the data to a remote storage location – represented conceptually as a cloud server or central hospital database 370 in the figure. The term “cloud-based recording” implies that the data is being sent to a secure server infrastructure where it can be archived long-term and accessed by authorized personnel later. This upload may happen continuously as the data is captured (streaming the data off-site as an extra backup and for live monitoring by quality assurance systems), or it might be done in batches (e.g., saving locally during surgery and then pushing the complete dataset to the cloud once the procedure is over, depending on bandwidth or hospital policy). In either case, the system uses secure protocols to protect this information: patient data and surgical video are highly sensitive, so encryption and compliance with privacy regulations (such as HIPAA) are enforced during transmission and in storage. The cloud-based approach has the advantage of making the data immediately available for remote analysis or consultation, even during the procedure – for instance, a specialist at a different facility could review the incoming video in near real time if needed for a second opinion, or automated algorithms in the cloud could start analyzing the data for certain patterns (like measuring durations of surgical steps for efficiency analysis).

Once data is being stored, step 650 involves managing and indexing the recorded data for both immediate and future use. The control system 160 (or the cloud backend) organizes the captured information into a structured format. This could mean compiling the video into segments labeled by phase of surgery (incision, closure, etc., if it can detect those), or simply ensuring all metadata annotations from step 630 are embedded as markers in the video file or in an accompanying log file. Step 650 may also include generating a summary or report of the procedure automatically. For example, at the end of recording, the system 100 could produce a brief report listing key events and timestamps: “Procedure Start: 10:30:00; Incision: 10:45:23; Instrument count discrepancy alert at 12:10:05; Procedure End: 12:30:00; Surgeon: Dr. Smith; Cobot recorded video length: 2 hours.” This kind of summary, along with the full detailed dataset, can be extremely valuable for postoperative review, training, or compliance audits. Moreover, because the data is richly annotated, someone reviewing later can jump to specific points of interest (for instance, directly to when a complication occurred, guided by the system’s marks).

In step 660, the process concludes with the data availability and optional analysis. The complete recorded dataset, which is stored in the cloud or a central database at this stage, becomes accessible to authorized users. Surgeons or surgical trainees may later replay the procedure using an interface that can even leverage VR: for example, they could re-experience the surgery in a virtual environment using the cobot’s recorded 3D video data, complete with the AR annotations that were made (or even add new annotations for teaching purposes). Hospital quality teams might analyze the timing data to identify workflow bottlenecks. Additionally, AI models could be trained on this accumulated data (with appropriate anonymization) to improve future recognition accuracy or to derive predictive analytics (e.g., correlating certain instrument usage patterns with outcomes). If any incidents or anomalies occurred during surgery, the detailed documentation captured by the cobot 100 serves as a “black box” for investigation and learning, something traditionally lacking in surgeries. Throughout this flow, an emphasis is placed on data security and integrity: checksums or redundant recordings might be used to ensure no frames are lost, and all data is time-synchronized to maintain a coherent record. By the end of the process in FIG. 6, the perioperative cobot system 100 has created a comprehensive, high-fidelity record of the surgical procedure, complete with contextual information. This fulfills the goal of intraoperative data acquisition and documentation in a manner that is unobtrusive (requiring little to no manual recording by staff), accurate, and immediately useful for improving surgical practice, training, and compliance.

In view of the foregoing, the perioperative humanoid cobot system 100 with integrated vision and telepresence provides an advanced, intelligent assistant for surgical environments. It embodies a synergy of hardware and software modules that allow it to see the operative field in great detail (via high-resolution, multi-modal cameras 140), understand what it sees (through recognition algorithms running on control system 160), communicate and extend the reach of the surgical team (via the multimodal communication interface 150 enabling telepresence and AR collaboration), and record everything reliably (through the data acquisition and cloud logging process of FIG. 6). The humanoid design with torso 110, head 120, and arms 130 ensures it can physically integrate into the OR much like a human team member, moving with autonomy but deference to surgical protocols, never violating sterile boundaries 340 and always prioritizing safety. The modular architecture allows the system to be configured for various perioperative roles, whether it be as a telepresence avatar for a remote specialist, a vigilant observer that can call attention to details, or a documentation system creating a rich surgical log.

While specific embodiments have been described to enable practice of the disclosed subject matter, they are illustrative and not limiting. Variations, modifications, substitutions, and combinations of the features described can be made by those skilled in the art without departing from the scope of this disclosure. Features described with respect to one embodiment may be used with, or substituted for, features of another. Unless expressly stated otherwise, the terms “comprising,” “including,” and “having” are used in an open, non‑limiting sense; the singular forms “a,” “an,” and “the” include the plural forms; and stated ranges include their endpoints and all subranges. Unless the context clearly indicates otherwise, “and/or” means one or more of the listed items (e.g., “A and/or B” encompasses A alone, B alone, or both A and B); “or” is inclusive (i.e., “A or B” means A, B, or both); and “and” is conjunctive. Headings and ordering are for convenience and do not limit the description. The scope of the claimed subject matter is defined solely by the claims that follow and their legal equivalents.

What is claimed is:

1. A perioperative humanoid collaborative robot system, comprising:
a humanoid robotic body including a torso and two articulated arms, with at least one camera mounted on an upper portion of the body selected from a head portion and the torso for capturing visual data;
a stereoscopic vision system comprising at least two image sensors configured to provide high-resolution, low-latency three-dimensional imaging of a surgical field;
a communication interface configured for real-time wireless data communication to enable remote pilot operation of the robot and telepresence interaction with surgical staff;
an audio communication module comprising at least one microphone and at least one speaker, the audio communication module configured to perform voice recognition of spoken commands from the surgical staff and to output synthesized speech to the surgical staff;
an instrument recognition module configured to scan a surgical instrument tray and identify surgical instruments for verifying an instrument inventory;
an environment recognition module configured to detect room layout features and recognize personnel within an operating room, and to provide data for augmented reality visualization to support remote expert instruction;
a data recording module configured to record surgical activity data including timestamped events and classified event types, and to store the data in a cloud-based storage system;
a safety system comprising one or more sensors and control logic configured to detect obstacles and enforce sterile field compliance by preventing the robot from entering predefined sterile zones in the operating room; and
a control system comprising at least one processor and memory, the control system configured to control autonomous or semi-autonomous navigation of the robot within the operating room and to coordinate data acquisition by the vision system, instrument recognition module, environment recognition module, and data recording module.

2. The system of claim 1, wherein the stereoscopic vision system comprises at least one camera that is at least one of telescopically extendable from the robot and detachably mountable to the robot to provide adjustable viewing angles.

3. The system of claim 1, wherein the stereoscopic vision system further comprises at least one thermal imaging sensor configured to capture infrared thermal imagery of the surgical environment in addition to optical images.

4. The system of claim 1, wherein the communication interface comprises a 5G cellular transceiver, a Wi-Fi transceiver, and a Bluetooth transceiver for multi-channel wireless connectivity.

5. The system of claim 1, wherein the audio communication module further comprises a noise-cancellation component configured to filter out ambient operating room noise to enhance voice recognition accuracy.

6. The system of claim 1, wherein the instrument recognition module is configured to perform an automated scan of a surgical instrument tray by capturing images of the surgical instruments on the tray and comparing the recognized instruments against a predetermined inventory list.

7. The system of claim 1, wherein the instrument recognition module comprises an artificial intelligence model trained on surgical instrument data to recognize and classify surgical instruments from visual data with a predetermined confidence threshold.

8. The system of claim 1, wherein the data recording module is configured to timestamp and categorize intraoperative events and to upload the recorded data to the cloud-based storage, and wherein the data recording module further utilizes artificial intelligence analysis of the recorded data to generate efficiency metrics for the surgical procedure.

9. The system of claim 1, wherein the safety system comprises a three-dimensional mapping sensor and is configured to create a map of the operating room including at least one sterile field boundary, and to automatically halt or adjust the robot’s movement upon detecting a potential collision or a breach of the sterile field boundary.

10. The system of claim 1, wherein each of the articulated arms of the humanoid robotic body is telescopically extendable to increase the arm’s reach and to facilitate adjustable positioning of sensors or instruments.

11. A surgical telepresence system, comprising:
a humanoid collaborative robot positioned in an operating room, the robot including a stereoscopic vision system and an audio communication module (with at least one camera, microphone, and speaker) for capturing real-time video and audio of the operating room; and
a remote operator station including a display device and a control interface, the remote operator station configured to receive the real-time video and audio from the robot and to transmit control signals and audio input to the robot;
wherein the humanoid robot and the remote operator station are communicatively linked via a wireless network to enable real-time telepresence interaction such that a remote expert at the remote operator station can view a stereoscopic representation of the operating room environment through the display device and communicate with the surgical staff via the humanoid robot.

12. The system of claim 11, wherein the wireless network link between the humanoid robot and the remote operator station comprises at least one of a 5G cellular connection and a Wi-Fi connection to provide low-latency, high-bandwidth transmission of the video and audio data.

13. The system of claim 11, wherein the remote operator station comprises a head-mounted display configured to present the remote expert with an immersive virtual reality view of the operating room from the robot’s stereoscopic vision system.

14. The system of claim 11, further comprising an augmented reality projection unit on the humanoid robot, the projection unit configured to project visual guidance cues or annotations into the operating room under control of the remote expert.

15. The system of claim 11, further comprising an environment recognition module on the humanoid robot configured to identify personnel and sterile zones in the operating room, wherein the remote operator station is configured to overlay indicators corresponding to the identified personnel and sterile zones onto the display device for the remote expert as augmented reality annotations.

16. The system of claim 11, wherein the humanoid robot is configured to operate in a teleoperation mode that allows the remote expert to remotely control the robot’s movements and adjust the robot’s camera viewpoints in real time via the control interface of the remote operator station.

17. A method of supporting a surgical procedure using a perioperative humanoid collaborative robot, the method comprising:
positioning the humanoid collaborative robot in an operating room such that a stereoscopic vision system of the robot has a view of a surgical field;
establishing a real-time communication link between the robot and a remote expert;
capturing stereoscopic video of the surgical field and audio of the operating room via the robot’s cameras and microphone;
transmitting the captured video and audio in real time to the remote expert over the communication link;
receiving instruction signals from the remote expert at the robot; and
relaying the remote expert’s instructions to the surgical staff in the operating room via at least one of audible output from the robot’s speaker or visual cues provided by the robot.

18. The method of claim 17, further comprising scanning, by the humanoid collaborative robot, a surgical instrument tray with the robot’s vision system to capture images of a plurality of surgical instruments.

19. The method of claim 17, further comprising identifying, from visual data captured by the humanoid collaborative robot, individual surgical instruments on a surgical instrument tray and verifying that each instrument of a predetermined required set is present.

20. The method of claim 17, further comprising overlaying visual annotations on the real-time video feed transmitted to the remote expert to provide an augmented reality view of the surgical environment, thereby highlighting objects or regions of interest in the operating room for the remote expert.
